(19) United States
(12) Patent Application Publication
(10) Pub. No.: US 2012/0253183 A1
(43) Pub. Date: Oct. 4, 2012

(54) IMAGE PROCESSING APPARATUS, OPHTHALMIC APPARATUS, AND OPHTHALMIC SYSTEM

(75) Inventor: Kenji Muto, Fujisawa-shi (JP)

(73) Assignee: CANON KABUSHIKI KAISHA, Tokyo (JP)

(21) Appl. No.: 13/432,362

(22) Filed: Mar. 28, 2012

(30) Foreign Application Priority Data
Mar. 31, 2011 (JP) 2011-079366

(51) Int. Cl. A61B 6/02; G06T 15/00

(52) U.S. Cl. 600/425; 345/419

(57) ABSTRACT

To reduce confirmation time by an examiner to increase the diagnosis efficiency, a medical system including a medical apparatus that acquires a tomographic image of an object to be examined using combined light generated by combining return light from the object to be examined to which measuring light is emitted and reference light corresponding to the measuring light includes a display control unit configured to control a display unit to sequentially display the tomographic images in the predetermined region on the display unit, and a changing unit configured to change a display time period for the tomographic images displayed in the predetermined region based on the number of the tomographic images to be displayed.

### BACKGROUND OF THE INVENTION

1. Field of the Invention

The present invention relates to an image processing apparatus used for ophthalmic care or the like, an ophthalmic system, and an ophthalmic apparatus.

2. Description of the Related Art

In recent years, optical coherence tomography (hereinafter, also referred to as OCT) apparatuses for acquiring tomographic images using the interference by low coherence light have been put into practical use.

The OCT apparatuses can acquire a tomographic image at a resolution around the wavelength of light emitted to an object to be examined. Consequently, the tomographic image of the object to be examined can be acquired at a high resolution. Especially, such OCT apparatuses are useful for ophthalmic apparatuses for acquiring a tomographic image of a retina which positions on a fundus oculi.

Japanese Patent Application Laid-Open No. 2007-117714 discusses a technique for superimposing an acquisition position of a two-dimensional tomographic image on an eye-fundus image so that the acquisition position of the acquired two-dimensional tomographic image on the fundus oculi can be recognized.

In this technique, in acquisition processing of a three-dimensional tomographic image of the fundus oculi, the fundus oculi of the subject eye is two-dimensionally scanned with measuring light. Consequently, during the acquisition operation of the three-dimensional tomographic image of the fundus oculi, if involuntary eye movement of the subject eye occurs, a positional deviation may be generated in the two-dimensional tomographic image.

In another case, if the measuring light is blocked due to a blink of the subject eye or a cataract in an anterior eye part of the subject eye, a dark two-dimensional tomographic image is captured. In order to check whether a dark tomographic image or a tomographic image containing a positional deviation exists, each of the plurality of tomographic images can be sequentially displayed. In such a case, if the number of the tomographic images to be displayed is too large, the time necessary for checking the images by the examiner increases. The increase in checking time is undesirable in view of the diagnosis efficiency.

### SUMMARY OF THE INVENTION

According to an aspect of the present invention, a medical system including a medical apparatus that acquires a tomographic image of an object to be examined using combined light generated by combining return light from the object to be examined to which measuring light is emitted and reference light corresponding to the measuring light includes a display control unit configured to control a display unit to sequentially display the tomographic images in the predetermined region on the display unit, and a changing unit configured to change a display time period for the tomographic images displayed in the predetermined region based on the number of the tomographic images to be displayed.

According to the present invention, in consequently displaying a plurality of tomographic images, a time period for displaying the tomographic images can be changed based on the number of the tomographic images to be displayed. Consequently, the check time for the examiner can be reduced and the diagnosis efficiency can be increased.

Further features and aspects of the present invention will become apparent from the following detailed description of exemplary embodiments with reference to the attached drawings.

### BRIEF DESCRIPTION OF THE DRAWINGS

The accompanying drawings, which are incorporated in and constitute a part of the specification, illustrate exemplary embodiments, features, and aspects of the invention and, together with the description, serve to explain the principles of the invention.

FIG. 1 illustrates an acquired image confirmation screen according to first and second exemplary embodiments.

FIGS. 2A and 2B illustrate units included in an ophthalmic system according to the first exemplary embodiment.

FIG. 3 is a flowchart illustrating processes for implementing the individual units in the ophthalmic system according to the first exemplary embodiment.

FIG. 4 illustrates an image quality evaluation index of a tomographic image according to the first exemplary embodiment of the present invention.

### DESCRIPTION OF THE EMBODIMENTS

Various exemplary embodiments, features, and aspects of the invention will be described in detail below with reference to the drawings.

The ophthalmic system (or ophthalmic apparatus) according to exemplary embodiments of the present invention can change a time period for displaying individual tomographic images based on the number of the tomographic images to be displayed in sequentially displaying the individual tomographic images. By the processing, the display time per image can be reduced when the number of the images to be displayed is large, and consequently, the confirmation time for the examiner can be reduced and the diagnosis efficiency can be increased.

In addition to the ophthalmic system, the exemplary embodiments of the present invention can be applied to medical systems or medical devices for observing an object to be examined such as the skin of a human body, for example, an endoscope.

The number of the images to be displayed is, for example, the number of acquired two-dimensional tomographic images. In a case where a three-dimensional tomographic image is to be acquired, the number of the images to be displayed is the number of main scanning lines of a scanning unit for scanning the fundus oculi of a subject eye with measuring light. Thereby, all of the acquired tomographic images can be checked immediately after the image acquisition.

All of the acquired tomographic images is not always to be checked, and the number of the images to be displayed may be the number of automatically selected tomographic images of a target portion in a central part in the acquired images.

The number of the images to be displayed may be the number of automatically selected tomographic images having a positional deviation due to an involuntary eye movement of the subject eye.

The number of the images to be displayed may be the number of automatically selected dark tomographic images captured when the measuring light is blocked due to a blink of the subject eye or a cataract in an anterior eye part of the subject eye.

As described above, the dark tomographic images, the tomographic images having a positional deviation, or the like are determined to be a tomographic image having an image capturing failure, and the determined tomographic image is selected. The confirmation efficiency for checking whether an image capturing failure exists in a tomographic image by the examiner can be increased by increasing the display time or decreasing the display speed of the tomographic image containing the image capturing failure.

The automatic selection method includes methods using image evaluation indexes described below, or the like. For example, a tomographic image whose value of the image evaluation index is lower than a predetermined value is selected as the tomographic image containing a positional deviation. The number of the images to be displayed may be the number of the selected images such as a tomographic image of a target portion, a dark tomographic image, a tomographic image containing a positional deviation, or the like selected by the examiner. The tomographic images may be selectively checked by the examiner.

By this operation, the confirmation time (display time) per tomographic image that is needed to be closely checked can be increased, or the display speed can be decreased. As a result, the diagnosis efficiency can be increased.

In a case where a tomographic image contains an image capturing failure, it is preferable to input that the three-dimensional tomographic image of this time is not good (NG), and capture a three-dimensional tomographic image again. In such a case, according to the exemplary embodiments of the present invention, the efficiency for checking whether a tomographic image contain an image capturing failure can be increased, and consequently, the overall image capturing time can be reduced. As a result, a burden on the subject eye can be reduced.

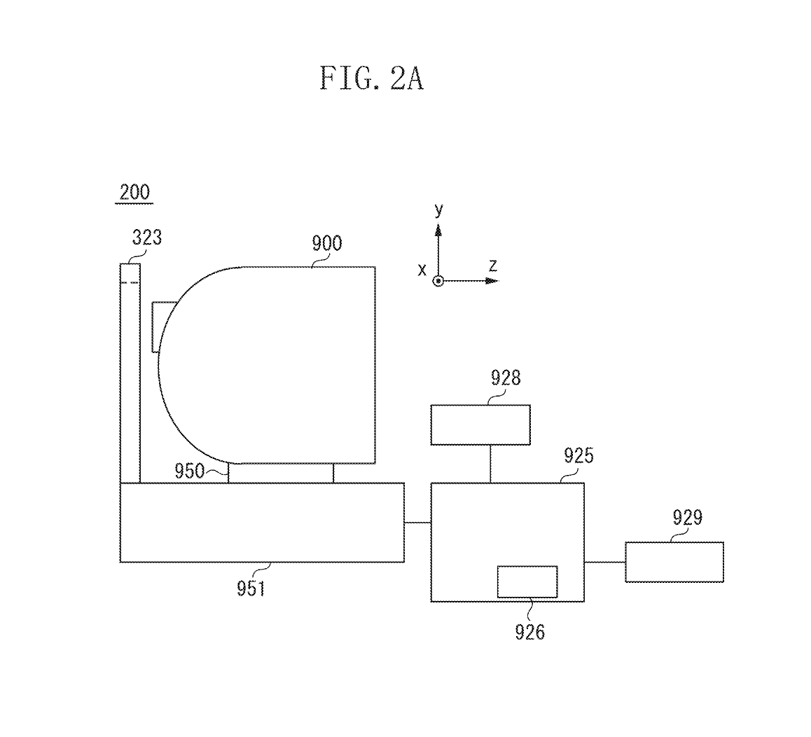

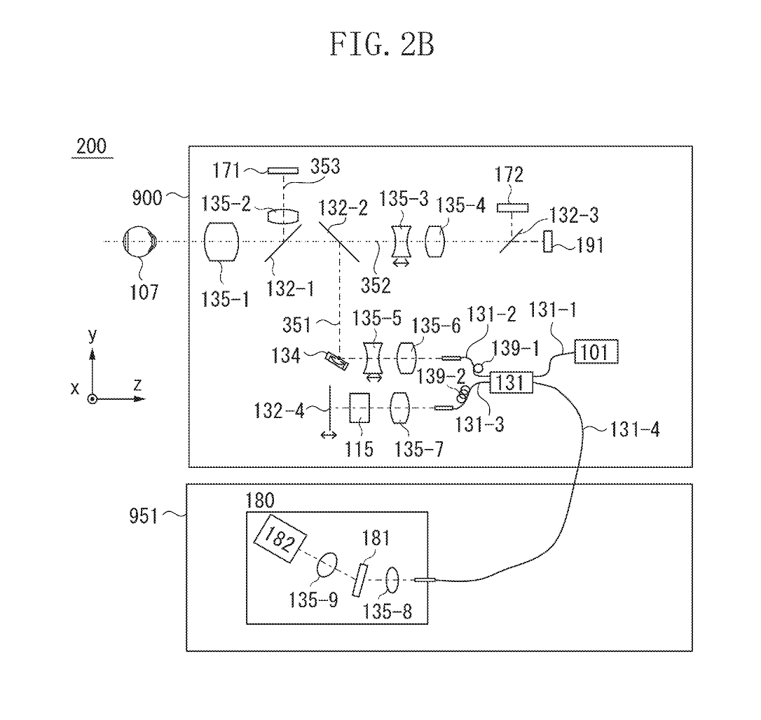

A schematic configuration of the ophthalmic apparatus according to the present exemplary embodiment is described with reference to FIG. 2A. FIG. 2A is a side view of the ophthalmic apparatus. An optical head 900 is a measurement optical system for acquiring an image of an anterior eye part, and a two-dimensional image and a tomographic image of a fundus oculi. The optical head 900 can move with respect to a base part 951 including a spectroscope, which will be described below, using a stage unit (also referred to as moving portion) 950 that is moved by a motor, or the like in the XYZ directions in FIG. 2A.

A personal computer 925 that also serves as a control unit of the stage unit 950 performs control of the stage unit and also forms a tomographic image. A hard disk 926 stores a program for tomographic image capturing and also serves as a subject information storage unit. A display control unit (not illustrated) instructs a display unit 928 such as a monitor to display an acquired image, or the like. An input unit 929 issues an instruction to the personal computer. Specifically, the input unit 929 includes a keyboard and a mouse (also referred to as pointing device). A chin rest 323 fixes the chin and forehead of a subject.

Configurations of the measurement optical system and the spectroscope in the ophthalmic apparatus according to the present exemplary embodiment are described with reference to FIG. 2B.

First, the internal configuration of the optical head 900 is described. An objective lens 135-1 is disposed opposite to a subject eye 107. On the optical axis, a first dichroic mirror 132-1 and a second dichroic mirror 132-2 split the light into wavelength bands of an optical path 351 for an OCT optical system, an optical path 352 for eye-fundus observation and a fixation lamp, and an optical path 353 for anterior eye part observation.

Similar to the above-described optical paths, the optical path 352 is further split by a third dichroic mirror 132-3 into wavelength bands of a charge coupled device (CCD) 172 for fundus observation and an optical path to a fixation lamp 191. The optical head 900 further includes lens 135-3 and lens 135-4. The lens 135-3 is driven by a motor (not illustrated) for the fixation lamp and for focus adjustment for eye-fundus observation.

The CCD 172 has sensitivity to a wavelength of eye-fundus observation illumination light (not illustrated), specifically, has sensitivity around 780 nm. The fixation lamp 191 emits visible light to urge the subject to fix his/her line of sight. The optical system for eye-fundus observation may include an optical system such as a scanning laser ophthalmoscope (SLO).

On the optical path 353, a lens 135-2 and an infrared CCD 171 for anterior eye observation are provided. The CCD 171 has sensitivity to a wavelength of anterior eye observation illumination light (not illustrated), specifically, has sensitivity around 970 nm. On the optical path 353, an image split prism (not illustrated) is disposed. Using the image split prism, a distance in the Z direction of the optical head 900 to the subject eye 107 can be detected as a split image in an anterior eye observation image.

The optical path 351, as described above, forms the OCT optical system for acquiring a tomographic image of the fundus oculi of the subject eye 107. More specifically, the optical path 351 is used for acquiring an interference signal for forming a tomographic image. An XY scanner (also referred to as scanning unit) 134 for scanning the fundus oculi with measuring light is illustrated as one mirror in FIG. 2B, however, the XY scanner is used for performing scan in the X-Y two axis direction.

The optical head 900 further includes lens 135-5 and lens 135-6. The lens 135-5 is driven by a motor (not illustrated) for focus adjustment of the light emitted from a light source 101 from a fiber 131-2 connected to an optical coupler 131 on the fundus oculi of the subject eye 107. By the focus adjustment operation, the light from the fundus oculi of the subject eye 107 simultaneously concentrated in a spotted state at the tip of the fiber 131-2 and entered.

Configurations of optical paths from the light source 101, a reference light optical system, and the spectroscope are described. In the present exemplary embodiment, a Michelson interference system is configured by including the light source 101, a mirror 132-4, a dispersion compensation glass 115, the optical coupler 131, single-mode optical fibers 131-1 to 131-4 which are integrally connected to the optical coupler, a lens 135-7, and a spectroscope 180.

The light emitted from the light source 101 passes through the optical fiber 131-1, and is split into measuring light at the optical fiber 131-2 side and reference light corresponding to the measuring light at the optical fiber 131-3 side via the optical coupler 131. The measuring light passes through the OCT optical system optical path, and the fundus oculi of the subject eye 107 which is the observation target is irradiated with the light. The light passes through the same optical path by reflection and scattering by the retina and reaches the optical coupler 131.

Meanwhile, the reference light passes through the optical fiber 131-3, the lens 135-7, and the dispersion compensation glass 115, which is inserted to compensate the dispersion of the measuring light and the reference light, and reaches the mirror 132-4. Then, the light is reflected by the mirror. The light returns via the same optical path and reaches the optical coupler 131.

The optical coupler 131 combines the measuring light and the reference light. The light is referred to as interference light or combined light. When the optical path length of the measuring light becomes substantially the same as the optical path length of the reference light, interference occurs. The mirror 132-4 is supported by a motor and driving mechanism (not illustrated) adjustably in the optical axis direction. The mirror 132-4 can adjust the optical path length of the reference light to the optical path length of the measuring light that varies due to the subject eye 107.

The interference light is guided to the spectroscope 180 via the optical fiber 131-4. A polarization adjustment unit 139-1 is provided at the measuring light side in the optical fiber 131-2. A polarization adjustment unit 139-2 is provided at the reference light side in the optical fiber 131-3.

These polarization adjustment units include parts formed by routing the optical fibers in a loop state. By turning the loop-state part centering the longitudinal direction of the fiber, the fiber is twisted, and as a result, the polarization state of the measuring light and the reference light can be adjusted respectively.

In the apparatus, the polarization state of the measuring light and the reference light is adjusted and fixed in advance. The spectroscope 180 includes lenses 135-8 and 135-9, a diffraction grating 181, and a line sensor 182. The interference light emitted from the optical fiber 131-4 passes through the lens 135-8 and converted into substantially parallel light. The light is separated by the diffraction grating 181, and forms an image on the line sensor 182 by the lens 135-3.

Next, the light source 101 is described in detail. The light source 101 is a super luminance diode (SLD) that is a typical low coherent light source. The central wavelength is 855 nm, and the bandwidth is about 100 nm.

The bandwidth has an effect on the resolution in the optical axis direction of the acquired tomographic image, and accordingly, an important parameter. As the light source, in the present exemplary embodiment, the SLD is selected. However, light sources that can emit low coherent light may be employed, for example, amplified spontaneous emission (ASE) may be employed.

With respect to the central wavelength, since an eye is the target of the measurement, infrared light is preferred. Moreover, since the central wavelength has an effect on the resolution in the horizontal direction of the acquired tomographic image, it is preferable to employ a short wavelength as much as possible. For the above-mentioned two reasons, the central wavelength of 855 nm is employed.

In the present exemplary embodiment, the Michelson interferometer is used for the interferometer, however, a Mach-Zehnder interferometer can be employed. When the light amount difference between the measuring light and the reference light is relatively small, the Michelson interferometer whose separation unit and combining unit are commonly provided is preferable to the Mach-Zehnder interferometer whose separation unit and combining unit are separately provided.

Next, the method of acquiring a tomographic image is described. A control unit (not illustrated) can acquire a tomographic image of a desired portion on the fundus oculi of the subject eye 107 by controlling an XY scanner 134. The XY scanner performs scanning in the X direction with measuring light 105. The line sensor 182 captures information of a predetermined number of scanned lines from an image capturing range in the X direction on the fundus oculi.

Then, Fast Fourier transformation (FFT) processing is performed on a luminance distribution on the line sensor 182, the luminance distribution is acquired at a position in the X direction. The linear luminance distribution acquired by the FFT processing is converted into density or color information in order to display the information on a monitor 928, and the information is called an A-scan image. A plurality of A-scan images are two-dimensionally arranged to form a two-dimensional image. The two-dimensional image is called a B-scan image.

A plurality of B-scan images are acquired by capturing a plurality of A-scan images to form one B-scan image, and then, moving the scanning position in the Y direction and performing scanning in the X direction again. The examiner sees the B-scan images displayed on the monitor 928 or a three-dimensional tomographic image formed from the B-scan images, and can conduct a diagnosis of the subject eye.

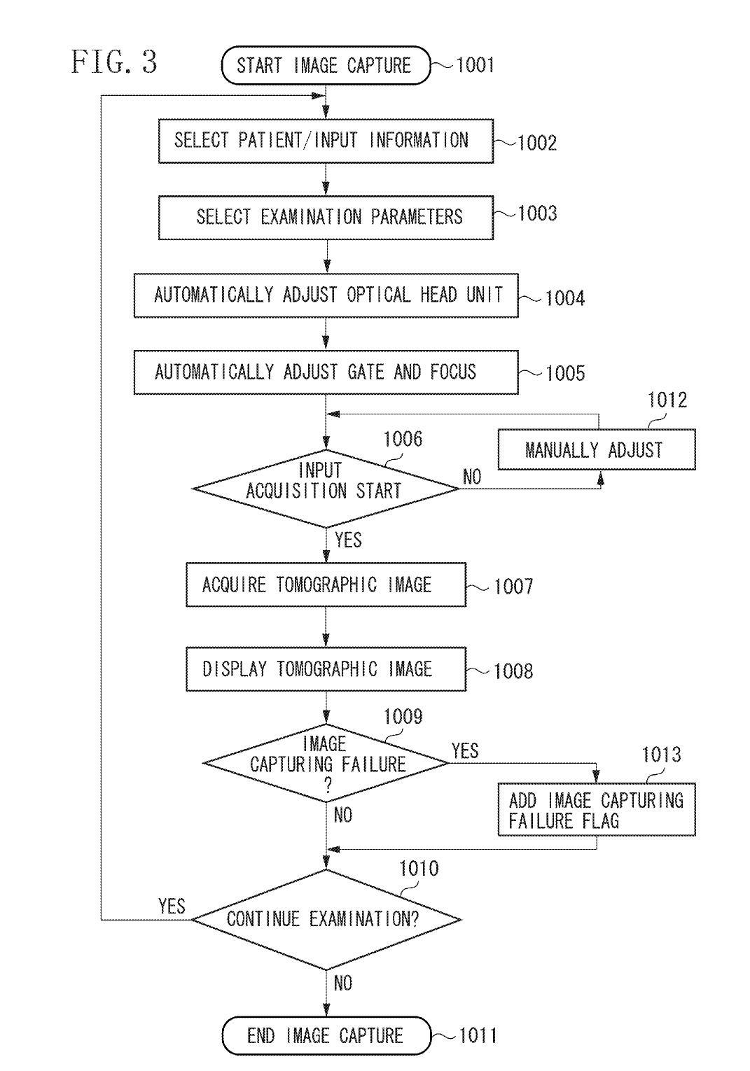

The processing implemented by the units in the ophthalmic system according to the present exemplary embodiment is described with reference to FIG. 3.

In step S1001, acquisition of a tomographic image is started. The personal computer 925 executes a program for acquiring tomographic images and instructs the monitor 928 to display an initial screen. Further, the personal computer 925 operates the XY scanner 134. Then, the processing automatically proceeds to step S1002.

In step S1002, the personal computer 925 instructs the monitor 928 to display a patient information entry screen. The examiner selects a patient or if it is the first visit, enters patient information. In response to an operation such as a click operation of an OK button displayed on the patient information entry screen with a mouse, the processing proceeds to step S1003.

In step S1003, the personal computer 925 instructs the monitor 928 to display an examination parameter selection screen. The examiner sets the examination parameters such as right or left of the subject eye, in which range a tomographic image is to be captured, how many times the tomographic image is to be captured, the number of A-scan images contained in a B-scan image, and the like.

The settings relating to the tomographic image capturing is called a scan pattern. In response to an operation of the examiner such as a click operation of an OK button displayed on an examination parameter selection screen with the mouse, the processing proceeds to step S1004.

In step S1004, the personal computer 925 instructs the optical head 900 to move to an initial alignment position. Then, the personal computer 925 instructs the optical head 900 to move to a measurement start position corresponding to right or left of the subject eye. Then, the personal computer 925 acquires an image of the anterior eye part of the subject eye 107 with the CCD 171 for anterior eye observation. With respect to the XY directions in FIG. 2A, the control unit moves the optical head 900 such that the center of the pupil which is to be an initial adjustment target position corresponds to an image central position.

With respect to the Z direction, the control unit adjusts the optical head 900 in the z direction so that a bright spot projected on the anterior eye part has a smallest size, and then, the processing automatically proceeds to step S1005. In step S1005, an eye-fundus image and a tomographic image are previewed on the monitor 928.

Simultaneously, in this step, the automatic operation of the optical path length adjustment of the reference optical path by the movement of the mirror 132-4, the focusing of the eye-fundus image by the lens 135-3, and the focusing of the tomographic image by the lens 135-5 are performed.

In step S1006, the personal computer 925 determines whether a signal for starting acquisition of a tomographic image is input by the examiner. If the personal computer 925 determines that the signal is input (YES in step S1006), the processing proceeds to step S1007. If the personal computer 925 determines that the signal is not input (NO in step S1006), the processing proceeds to step S1012. In step S1012, in order to further improve the automatically performed adjustment, an adjustment instruction can be manually issued. After the manual adjustment, again, the personal computer 925 waits for an input of the signal for starting acquisition of a tomographic image.

In step S1007, the personal computer 925 acquires a tomographic image using the scan pattern set in step S1003. Then, the personal computer 925 stores the tomographic image and an eye-fundus image acquired by the CCD for eye-fundus observation in a storage device in the personal computer 925, and the processing automatically proceeds to step S1008.

The storage operation may be automatically performed or may be performed by clicking an image capturing button displayed on the screen with the mouse. In step S1008, in order to determine whether the acquired tomographic image contain an image capturing failure, for the examiner, the acquired tomographic image is displayed on an acquisition image confirmation screen 2000, which is described below.

In step S1009, the personal computer 925 waits for an input of a signal relating to existence of an image capturing failure in the tomographic image. If the examiner determines that the tomographic image does not contain an image capturing failure (NO in step S1009), the examiner performs a click operation of an OK button 2214 on the screen with the mouse, or the like. Then, the processing proceeds to step S1010.

If the examiner determines that the tomographic image contains an image capturing failure (YES in step S1009), the examiner performs a click operation of a NG button 2213 on the screen with the mouse, or the like. Then, the processing proceeds to step S1013. In step S1013, the examiner adds (selects the tomographic image containing the image capturing failure) a flag indicating the image capturing failure to the stored data of the tomographic image. Then, the processing automatically proceeds to step S1010.

The flag indicates that the tomographic image contains the image capturing failure when the tomographic image is read and displayed again. In step S1010, the personal computer 925 issues instruction to display a screen for selecting whether to continue the examination or end the examination. The examiner selects one of the choices. If the examiner selects to continue the examination (YES in step S1010), the processing proceeds to step S1003. If the examiner selects to end the examination (NO in step S1010), the processing proceeds to step S1011, and ends the examination.

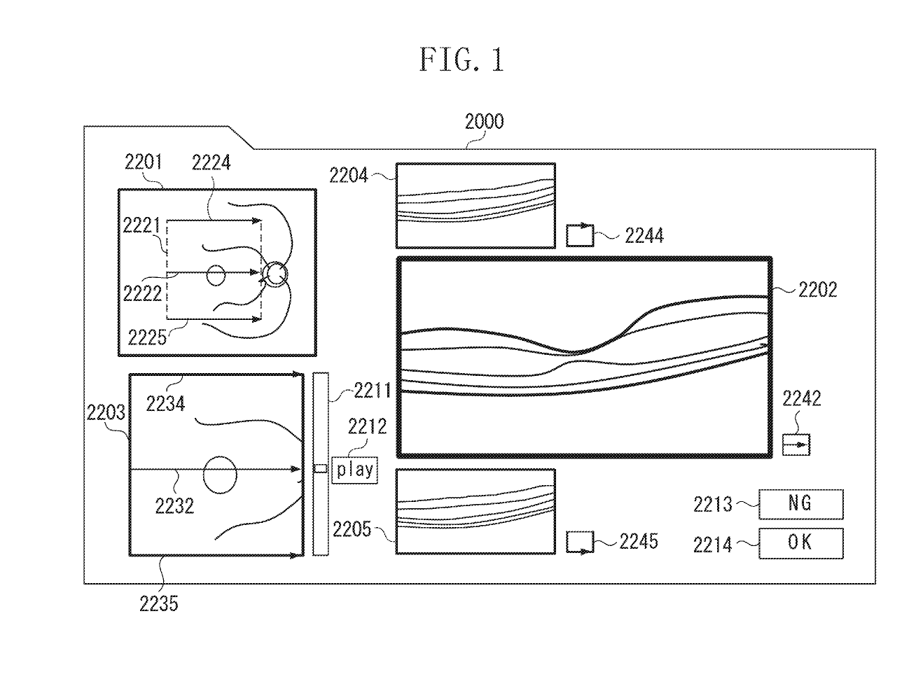

The above-described acquired image confirmation screen 2000 is described with reference to FIG. 1. A slider 2211 specifies a cross-sectional point of the tomographic image displayed on a main screen (also referred to as a first region) 2202 for displaying the tomographic image to check the acquired tomographic image.

A region (also referred to as a third region) 2242 schematically illustrates the acquisition position of the tomographic image in the eye-fundus image displayed on the main screen 2202. Regions (also referred to as third regions) 2244 and 2245 schematically illustrate acquisition positions of tomographic images in eye-fundus images displayed on sub screens 2204 and 2205. The sub screens 2204 and 2205 display tomographic images whose levels of importance are relatively lower than that of the tomographic image displayed on the main screen.

A display screen (also referred to as second region) 2201 displays the eye-fundus two-dimensional image (also referred to as an eye-fundus image) acquired by the CCD for eye-fundus observation. On the display screen 2201, a tomographic image acquisition range 2221, and arrows 2222, 2224, and 2225 are displayed. The arrows indicate positions (also referred to as acquisition positions on the eye-fundus image, or scanning positions of the scanning unit) and scanning directions in the tomographic image acquisition range 2221 of the tomographic image displayed on the main screen 2202, the sub screen 2204, or the sub screen 2205.

A screen (also referred to a C-scan screen) 2203 displays an eye-fundus image newly formed from the acquired tomographic image. On the screen 2203, arrows 2232, 2234, and 2235 indicating positions and scanning directions in the tomographic image acquisition range 2221 of the tomographic image displayed on the main screen 2202, the sub screen 2204, or the sub screen 2205, and the scanning directions are displayed.

In an initial state of the acquisition image confirmation screen 2000, on the main screen 2202, a tomographic image at a central position in the tomographic image acquisition range 2221 is displayed, on the screen 2204, a tomographic image at one end part position in the tomographic image acquisition range 2221 is displayed, and on the screen 2205, a tomographic image at the end part position opposite to the side displayed on the screen 2204 in the tomographic image acquisition range 2221 is displayed.

Using the tomographic image at the central position and the tomographic images at the both end parts, the examiner can roughly determine whether the tomographic images within the tomographic image acquisition range are displayed within the screens. Since an eyeball has a spherical shape, and especially, the radius of the spherical surface of a subject of excessive myopia is small, it is important to check the tomographic images at end parts and a central part.

If the examiner wants to check each tomographic image more closely, the examiner clicks a play button 2212 displayed on the acquisition image confirmation screen 2000 with the mouse, or the like. By the operation, on the main screen 2202, all tomographic images within the tomographic image acquisition range can be sequentially displayed.

More specifically, the tomographic images from the end part displayed on the screen 2204 to the other end part displayed on the screen 2205 are displayed one by one. The images are repeatedly displayed. The operation enables the examiner to check all tomographic images.

The display time for one tomographic image repeatedly displayed on the main screen is automatically changed depending on the number of the acquired tomographic images (also referred to as the number of main scanning lines of the scanning unit). The time for displaying all the tomographic images once can be set to a certain period of time, for example, two seconds or three seconds, to enable the examiner to check all the tomographic images in a short time.

Further, in the image display on the main screen, the display time for one image can be increased with respect to only a tomographic image important for determining whether an image capturing failure is contained, or only the image and its neighboring images. The apparatus determines, based on the acquired plurality of tomographic images, the importance of the tomographic images, and selects a tomographic image. The operation enables the examiner to further properly check the existence of an image capturing failure.

The above-described selected tomographic image include, for example, the following tomographic images.

A tomographic image whose image quality is high, and its neighboring tomographic images are to be displayed for a period of time longer than that of the other tomographic images. The examiner can focus on these tomographic images, and consequently, the captured image states such as layer separation can be checked more closely.

An evaluation index of the image quality is one of OCT image evaluation indexes, for example, the evaluation index may be a Q index indicating a ratio of pixels effective for diagnosis in a histogram of an image. A program for calculating the Q index and compares the Q index with a target value or a value at another alignment position corresponds to an image comparison unit according to the exemplary embodiment.

The program is integrated into the above-described program for image capture, and to be executed in the personal computer 925 that functions as an apparatus control unit. A method of calculating the Q index is described in British Journal of Ophthalmology 2006; 90: P186-190 “A new quality assessment parameter for optical coherence tomography”.

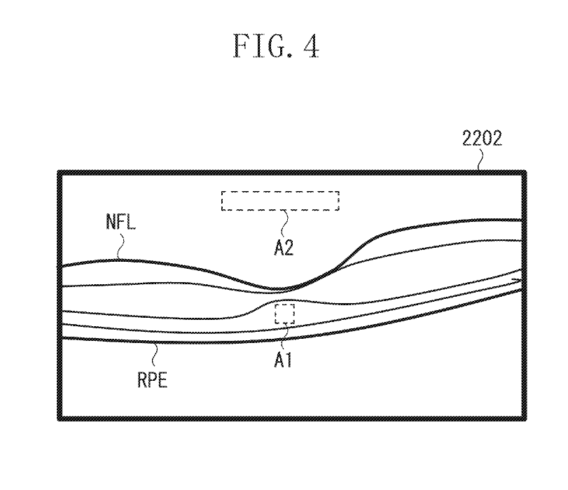

In the present exemplary embodiment, the Q index value is used as the guide of the image quality. Alternatively, the following image evaluation indexes may be employed. For example, a signal-to-noise ratio (SNR) indicating a ratio of a maximum value of a luminance value in an image to a luminance value of a background noise may be employed. Alternatively, a local image contrast that can be calculated from an average luminance value of a local region in a retina and an average luminance value of a background may be employed. The contrast is described with reference to FIG. 4.

FIG. 4 illustrates a preview screen of the tomographic image displayed on the screen 2202. The region A1 is a part of the olfactory nerve layer (ONL) (external granular layer) that is relatively dark in the retinal layers. The region A2 is a part of the background part. The contrast is calculated using average luminance values in the two regions.

Instead of the local contrast of the ONL and the background, a contrast between layers necessary for a diagnosis, or a contrast between a layer and the background may be employed, or the apparatus may be set so that the examiner selects a contrast. For the local image contrast calculation, segmentation for identifying the ONL or the like and recognize the regions is to be provided.

In acquisition of a tomographic image, in many cases, a target position such as the macula retinae or the optic papilla is determined in advance using a scanning pattern. In such a case, image capturing operation is performed by leading the macula retinae or the optic papilla in a central part of the image capture range using the fixation lamp. However, depending on the subject, the individual values may not position within the image capturing range.

To solve such a problem, not the tomographic image at the central position, but a tomographic image captured by passing through the macula retinae center or the optic papilla center determined by image processing from a plurality of captured tomographic images may be displayed for longer time. As a result, the examiner can check the target tomographic image and determine whether an image capturing failure is contained.

In a case where the measuring light is blocked due to a blink of the subject eye or a cataract in an anterior eye part of the subject eye, a dark tomographic image is captured. In another case, if involuntary eye movement of the subject eye occurs, a positional deviation may be generated in the tomographic image. As described above, the dark tomographic image, the tomographic image having the positional deviation, or the like are determined to be a tomographic image having an image capturing failure.

The confirmation efficiency for checking whether an image capturing failure exists in a tomographic image by the examiner can be increased by increasing the display time or decreasing the display speed of the tomographic image containing the image capturing failure.

In this processing, a tomographic image whose image evaluation index is low is to be selected. When the examiner determines whether the tomographic image contains an image capturing failure, if a plurality of tomographic images contain image capturing failures, in the sequential display of the acquired tomographic images on the main screen 2202, the tomographic images may be stationarily displayed on the sub screens 2204 and 2205.

In the stationary display, the two sub screens or more sub screens may be used. Further, in the stationary display, for example, out of selected tomographic images, a tomographic image whose image evaluation index value is lowest may be preferentially stationarily displayed on a sub screen.

An ophthalmic system or an ophthalmic apparatus according to a second exemplary embodiment differs from that in the first exemplary embodiment in that a tomographic image containing an image capturing failure or the like is stationarily displayed on the main screen 2202 after all acquired tomographic images are sequentially displayed. The processing enables the examiner to easily determine whether the acquired tomographic images contain an image capturing failure.

Descriptions of parts similar to those in the above-described first exemplary embodiment are omitted. During the stationary display, the play button 2221 is in the standby state again, and the sequential display can be performed again to enable the examiner to check the tomographic images again. The above-described important tomographic image includes, for example, the following tomographic images. Similar to the first exemplary embodiment, the apparatus determines the importance of the tomographic images based on the plurality of acquired tomographic images, and selects a tomographic image.

The tomographic images displayed before the play button 2212 was clicked again are displayed again. This operation is performed since the macula retinae center or the optic papilla center that is the target position is likely to be located near the central position by the guide by the fixation lamp.

(1) High Quality Tomographic Image

The point that the examiner can focus on those tomographic images, and can more closely check the image capture states such as layer separation is similar to that in the first exemplary embodiment. With respect to the image evaluation indexes, the image evaluation indexes are similar to those in the first exemplary embodiment and accordingly, their description is omitted.

(2) Tomographic Image Display in Which Lesion is Detected

A tomographic image determined to have a lesion or suspected to have a lesion by the apparatus is further directly and stationarily displayed. For example, in a case of glaucoma, a tomographic image in which a nerve fiber layer (NFL) is determined to be thin by segmentation corresponds to the above-mentioned tomographic image. The examiner sees the tomographic image, and can determine whether the tomographic image contains an image capturing failure and use the tomographic image as a reference for diagnosis. Further, setting of stationary display of a particular lesion enables the examiner to easily check whether the lesion that was previously diagnosed of a subject who visits for diagnosis again is properly captured.

(3) Tomographic Image of Target Position (Macula Retinae Center or Optic Papilla Center)

A tomographic image of a target position that is defined by a scan pattern such as the macula retinae center or the optic papilla center is displayed. The examiner can easily determine whether the tomographic image includes an image capturing failure by stationarily displaying the image of the target position.

(4) Tomographic Image Determined to Have Image Capturing Failure

A tomographic image determined to have an image capturing failure similar to the first exemplary embodiment is selected and stationarily displayed. Consequently, the examiner can further directly determine whether the tomographic image contains an image capturing failure. In a case where a plurality of tomographic images exist in determining the existence of an image capturing failure, the images may be stationarily displayed on the sub screens 2204 and 2205.

In such a case, the sub screens are not limited to the two screens, more screens may be used to display the images. In a case where a more important tomographic image in the selected plurality of images, for example, the image containing an image capturing failure in the example (3) is to be stationarily displayed, a tomographic image having a lower image index may be preferentially stationarily displayed on a sub screen.

Aspects of the present invention can also be realized by a computer of a system or apparatus (or devices such as a CPU or MPU) that reads out and executes a program recorded on a memory device to perform the functions of the above-described embodiments, and by a method, the steps of which are performed by a computer of a system or apparatus by, for example, reading out and executing a program recorded on a memory device to perform the functions of the above-described embodiments. For this purpose, the program is provided to the computer for example via a network or from a recording medium of various types serving as the memory device (e.g., computer-readable medium). In such a case, the system or apparatus, and the recording medium where the program is stored, are included as being within the scope of the present invention.

While the present invention has been described with reference to exemplary embodiments, it is to be understood that the invention is not limited to the disclosed exemplary embodiments. The scope of the following claims is to be accorded the broadest interpretation so as to encompass all modifications, equivalent structures, and functions.

This application claims priority from Japanese Patent Application No. 2011-079366 filed Mar. 31, 2011, which is hereby incorporated by reference herein in its entirety.

What is claimed is:

1. An image processing apparatus comprising:
a display control unit configured to control a display unit to sequentially display a plurality of tomographic images of a subject eye in a predetermined region on the display unit, and to sequentially display a plurality of positions on a fundus image of the subject eye, wherein each of the plurality of positions is corresponding to an acquisition position where a tomographic image displayed in the predetermined region are acquired on a fundus of the subject eye; and
a changing unit configured to change a display time period for each of the plurality of tomographic images based on the number of the plurality of tomographic images to be displayed.

2. The image processing apparatus according to claim 1, wherein the display control unit controls the display unit to display a substantially central two-dimensional tomographic image in the predetermined region in three-dimensional tomographic images formed from the plurality of tomographic images, to display two tomographic images at end parts in the three-dimensional tomographic images in regions smaller than the predetermined region and positioned above and below the predetermined region, and to sequentially display the tomographic images in the acquisition order in the predetermined region in response to a signal for instructing to sequentially display the tomographic images.

3. The image processing apparatus according to claim 1, further comprising:
a changing unit configured to change a display time period for displaying the tomographic images being displayed in the predetermined region and a position corresponding to the acquisition position of the tomographic images based on the number of the tomographic images to be displayed.

4. The image processing apparatus according to claim 1, wherein the number of the tomographic images to be displayed is the number of main scanning lines of measurement light on the fundus oculi of the subject eye.

5. The image processing apparatus according to claim 1, further comprising:
a selection unit configured to select at least one tomographic image out of the plurality of tomographic images,
wherein, the display time period of the tomographic images to be displayed in the predetermined region is changed based on the number of selected tomographic images selected by the selection unit.

6. The image processing apparatus according to claim 5, further comprising:
a calculation unit configured to calculate an image evaluation index for each of the plurality of tomographic images,
wherein the selection unit selects a tomographic image having a lower image evaluation index out of the plurality of tomographic images.

7. A medical system including a medical apparatus that acquires a tomographic image of an object to be examined using combined light generated by combining return light from the object to be examined to which measuring light is emitted and reference light corresponding to the measuring light, the medical system comprising:
a display control unit configured to control a display unit to sequentially display the tomographic images in the predetermined region on the display unit; and
a changing unit configured to change a display time period for the tomographic images displayed in the predetermined region based on the number of the tomographic images to be displayed.

8. A medical system including a medical apparatus that acquires a tomographic image of an object to be examined using combined light generated by combining return light from the object to be examined to which measuring light is emitted and reference light corresponding to the measuring light, the medical system comprising:
a display control unit configured to control a display unit to sequentially display the tomographic images in a first region on the display unit, and display a position corresponding to an acquisition position of the tomographic images being displayed in the first region on an eye-fundus image of the object to be examined displayed in a second region on the display unit.

9. The medical system according to claim 8, further comprising:
a changing unit configured to change a display time period for displaying the tomographic images being displayed in the first region and the position corresponding to the acquisition position of the tomographic images based on the number of the tomographic images to be displayed.

10. The medical system according to claim 8, further comprising:
a scanning unit configured to scan the object to be examined with the measuring light,
wherein a scanning position and a scanning direction of the scanning unit in the acquisition of the tomographic image displayed in the first region are arranged and displayed in a third region smaller than the first region and positioned in a vicinity of the first region.

11. A non-transitory computer-readable storage medium storing a program for causing a computer to implement the individual functions of the image processing apparatus according to claim 1.

12. An ophthalmic apparatus comprising:
a tomographic image acquisition unit configured to acquire tomographic images of a subject eye using combined light generated by combining return light from the subject eye to which measuring light is emitted and reference light corresponding to the measuring light;
a display control unit configured to control a display unit to sequentially display the tomographic images in a predetermined region on the display unit, and to display a position corresponding to a position where the tomographic images sequentially being displayed in the predetermined region are acquired over an eye-fundus image of the subject eye; and
a changing unit configured to change a display time period for the tomographic images displayed in the predetermined region based on the number of the tomographic images to be displayed.
